(19) United States
(12) Patent Application Publication
(10) Pub. No.: US 2024/0096477 A1
(43) Pub. Date: Mar. 21, 2024

(54) C-ARM IMAGING SYSTEM AND MEDICAL IMAGE PROCESSING METHOD

(71) Applicant: GE Precision Healthcare LLC, Wauwatosa, WI (US)

(72) Inventor: Xiang Li, Beijing (CN)

(21) Appl. No.: 17/948,038

(22) Filed: Sep. 19, 2022

(30) Foreign Application Priority Data
Sep. 18, 2022 (CN) 202111110379.8

(51) Int. Cl. G16H 30/40; A61B 6/00

(52) U.S. Cl. CPC G16H 30/40; A61B 6/4441

(57) ABSTRACT

Provided in embodiments of the present application are a C-arm imaging system and a medical image processing method. The medical image processing method includes acquiring medical images by using the C-arm imaging system; in a first stitching mode, labeling medical images selected from the acquired medical images, and stitching the labeled medical images to obtain a stitched image; and respectively displaying the acquired medical images, the labeled medical images, and the stitched image. According to the embodiments of the present application, an image stitching function suitable for a C-arm imaging system can be provided for existing C-arm imaging systems, thereby providing reference for a doctor to evaluate surgical effects during or after surgery.

### CROSS REFERENCE TO RELATED APPLICATIONS

This application claims priority to Chinese Patent Application No. 202111110379.8, filed on Sep. 18, 2021, the disclosure of which is incorporated herein by reference in its entirety.

### BACKGROUND

Embodiments of the present application relate to the technical field of medical devices, and particularly relate to a C-arm imaging system and a medical image processing method.

Orthopedists tend to use a panoramic image to assess surgical effects without delay in scoliosis surgery or the like. Typically, a doctor acquires a plurality of zone images during surgery and then acquires the panoramic image from DR (digital radiography) after the surgery.

Current DR systems can achieve a stitching function for radiological medical images. However, in the image stitching function of DR, firstly, it is necessary to determine the starting point and the ending point of a region of interest (ROI) of a patient, and control a x-ray tube to take a picture of the patient at a preset speed and a preset interval, i.e., the x-ray tube needs to move from the starting point to the ending point at a preset speed and to perform exposure and photography at an interval, and the moving velocity and/or location of the x-ray tube can be controlled based on a controller and/or the driving of a movement control mode of the x-ray tube (or a x-ray tube hanging bracket). But such a workflow is not suitable for C-arm imaging systems, and complex operations or separate interfaces can take too much time for the surgeon, thereby reducing efficiency during surgery.

It should be noted that the above description of the background is only for the convenience of clearly and completely describing the technical solutions of the present application, and for the convenience of understanding of those skilled in the art.

### SUMMARY

In view of at least one of the above technical problems, embodiments of the present application provide a C-arm imaging system and a medical image processing method. It is desirable to provide an image stitching function suitable for a C-arm imaging system for existing C-arm imaging systems, thereby providing reference for a doctor to evaluate surgical effects during or after surgery.

According to one aspect of the present application, a C-arm imaging system is provided, the system comprising an acquisition unit for acquiring medical images; a first stitching unit for labeling, in a first stitching mode, medical images selected from the acquired medical images, and stitching the labeled medical images to obtain a stitched image; and a display unit for respectively displaying the acquired medical images, the labeled medical images, and the stitched image.

In some embodiments, stitching the labeled medical images by the first stitching unit comprises determining whether stitching can be completed according to a preset condition; if it is determined that stitching can be completed, generating the stitched image.

In some embodiments, stitching the labeled medical images by the first stitching unit further comprises if it is determined that stitching cannot be completed, determining whether to acquire medical images again; if it is determined that medical images are acquired again, labeling medical images re-selected from the acquired medical images and stitching the re-labeled medical images.

In some embodiments, the first stitching unit labels the selected medical images according to the selecting order of the medical images and stitches the labeled medical images according to the labeling order.

In some embodiments, the apparatus further comprises a second stitching unit for stitching, in a second stitching mode, one or more medical images acquired in real time by the acquisition unit according to the acquiring order, so as to obtain a stitched image.

In some embodiments, the second stitching unit further labels the stitched medical images.

In some embodiments, stitching, by the second stitching unit, the medical images acquired in real time comprises determining whether stitching can be completed according to a preset condition; if it is determined that stitching can be completed, generating the stitched image.

In some embodiments, stitching, by the second stitching unit, the medical images acquired in real time further comprises if stitching ends, outputting the stitched image; and if stitching does not end, stitching medical images acquired again in real time.

In some embodiments, the apparatus further comprises a control unit for performing switching control to switch between the first stitching mode and the second stitching mode.

In some embodiments, the display unit displays the stitched image in a first region, and displays the acquired medical images and the labeled medical images in a second region as thumbnail images.

According to another aspect of the present application, a medical image processing method is provided and applied to a C-arm imaging system, the method comprising acquiring medical images by using the C-arm imaging system; in a first stitching mode, labeling medical images selected from the acquired medical images, and stitching the labeled medical images to obtain a stitched image; and respectively displaying the acquired medical images, the labeled medical images, and the stitched image.

According to another aspect of the present application, an electronic device is provided and comprises a memory and a processor, the memory storing a computer program, and the processor being configured to execute the computer program so as to implement the above-mentioned medical image processing method.

According to another aspect of the present application, a method for using a C-arm imaging system is provided, the method comprising moving the C-arm imaging system such that the C-arm imaging system acquires medical images at a plurality of positions; operating a stitching mode selecting button to select a stitching mode; and operating a stitching button such that the C-arm imaging system stitches the acquired medical images to obtain a stitched image and displays the acquired medical images and the stitched image.

One of the beneficial effects of the embodiments of the present application is that according to the embodiments of the present application, an image stitching function suitable for a C-arm imaging system can be provided for C-arm imaging systems, thereby providing reference for a doctor to evaluate surgical effects during or after surgery.

With reference to the following description and accompanying drawings, specific implementations of the embodiments of the present application are disclosed in detail, and manners in which the principle of the embodiments of the present application is employed are illustrated. It should be understood that the implementations of the present application are not thereby limited in scope. Within the spirit and scope of the appended claims, the implementations of the present application comprise various changes, modifications, and equivalents.

### BRIEF DESCRIPTION OF THE DRAWINGS

One of the beneficial effects of the embodiments of the present application is that according to the embodiments of the present application, an image stitching function suitable for a C-arm imaging system can be provided for C-arm imaging systems, thereby providing reference for a doctor to evaluate surgical effects during or after surgery.

The accompanying drawings are included to provide further understanding of embodiments of the present application, constitute a part of the specification, and are used to illustrate implementations of the present application and set forth the principles of the present application together with textual description. Obviously, the accompanying drawings in the following description are merely some embodiments of the present application, and a person of ordinary skill in the art could obtain other implementations according to the accompanying drawings without the exercise of inventive effort. In the accompanying drawings:

FIG. 1 is a schematic diagram of a C-arm imaging system according to an embodiment of the present application;

FIG. 2 is a schematic diagram of a control system for controlling the C-arm imaging system as shown in FIG. 1 according to an embodiment of the present application;

FIG. 3 is a schematic diagram of a medical image processing method according to an embodiment of the present application;

FIG. 4 is a flow diagram of a stitching process in a first stitching mode;

FIGS. 5A-5C are schematic diagrams of the stitching process in the first stitching mode;

FIG. 6 is a flow diagram of a stitching process in a second stitching mode;

FIGS. 7A-7D are schematic diagrams of the stitching process in the second stitching mode;

FIG. 8 is another schematic diagram of the C-arm imaging system according to an embodiment of the present application; and

FIG. 9 is a schematic diagram of an electronic device according to an embodiment of the present application.

### DETAILED DESCRIPTION

One of the beneficial effects of the embodiments of the present application is that according to the embodiments of the present application, an image stitching function suitable for a C-arm imaging system can be provided for C-arm imaging systems, thereby providing reference for a doctor to evaluate surgical effects during or after surgery.

The foregoing and other features of the embodiments of the present application will become apparent from the following description with reference to the accompanying drawings. In the description and the accompanying drawings, specific implementations of the present application are specifically disclosed, and part of the implementations in which the principles of the embodiments of the present application may be employed are indicated. It should be understood that the present application is not limited to the described implementations. On the contrary, the embodiments of the present application include all modifications, variations, and equivalents falling within the scope of the appended claims.

In the embodiments of the present application, the terms “first,” “second,” etc. are used to distinguish different elements, but do not represent a spatial arrangement or temporal order etc. of these elements, and these elements should not be limited by these terms. The terms “and/or” and “I” include any one of and all combinations of one or more of the associated listed terms. The terms “comprise,” “include,” “have,” etc. refer to the presence of described features, elements, components, or assemblies, but do not exclude the presence or addition of one or more other features, elements, components, or assemblies.

In the embodiments of the present application, the singular forms “a,” “the,” etc. include plural forms, and should be broadly construed as “a type of” or “a class of” rather than limited to the meaning of “one.” Furthermore, the term “said” should be construed as including both the singular and plural forms, unless otherwise specified in the context. In addition, the term “according to” should be construed as “at least in part according to . . . ,” and the term “based on” should be construed as “at least in part based on . . . ,” unless otherwise specified in the context.

The features described and/or illustrated for one implementation may be used in one or more other implementations in the same or similar manner, combined with features in other implementations, or replace features in other implementations. The term “include/comprise” when used herein refers to the presence of features, integrated components, steps, or assemblies, but does not preclude the presence or addition of one or more other features, integrated components, steps, or assemblies.

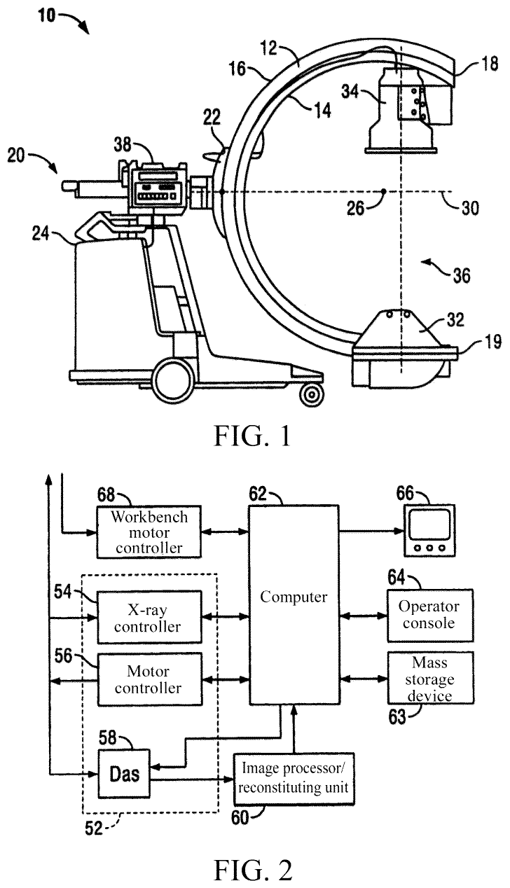

FIG. 1 is a schematic diagram of a C-arm imaging system according to an embodiment of the present application, schematically showing the appearance of a C-arm imaging system 10. As shown in FIG. 1, the C-arm imaging system 10 includes a C-arm support 12 having an inner circumference 14 and an outer circumference 16 and terminating at opposite upper and lower ends 18 and 19. In an exemplary embodiment, the C-arm support 12 has a uniform C-shape, but the present application is not limited thereto, and the C-arm support 12 may also include any arc-shaped member. Further, other configurations of a mobile imaging system may also be provided, for example, support arms may be positioned on the patient's bed, etc.

The C-arm support 12 is held at a suspension position by, for example, a support component such as a support member 20 that can include a support arm 22 mounted on a wheeled base 24. The support arm 22 is rotatably mounted relative to the wheeled base 24, e.g., by using a bearing device (not shown) between the support arm 22 and the C-arm support 12, or by the support arm 22 itself, thereby enabling rotational movement of the C-arm support 12 around a side spin shaft 30.

The wheeled base 24 enables the C-arm imaging system 10 to be moved from a first position to a second position, for example, from a first photographing position of a patient to a second photographing position of the patient to provide desired medical images for achieving image stitching. In the same way, wheels of the wheeled base 24 are used as transport components coupled to the support member 20, and for example, used for moving the support arm 22 and the C-arm support 12 from the first position to the second position when it may be desirable to move the C-arm imaging system 10 from one room to another room. Due to the movement properties of the C-arm imaging system 10 provided by the wheeled base 24, for example, more opportunities for use by patients in many different sickrooms in a hospital are provided.

The support arm 22 is slidably mounted on the outer circumference 16 of the C-arm support 12, the support member 20 includes a component, such as a structure and a mechanical device, such that the C-arm support 12 can perform selective sliding rail motion around a rail rotating shaft 26 to the selected position. The rail rotating shaft 26 may coincide with the center of curvature of the C-arm support 12, and coincide with the side spin shaft 30. It should be understood that due to the sliding rail motion, the C-arm support 12 moves to pass through various sliding points to a connection piece (not shown) of the support arm 22. The support member 20 further includes a component such as a mechanical device for rotating the support arm 22 around the side spin shaft 30 in the lateral direction by an optional amount to reach the selected lateral position. Due to combination of the sliding rail motion with side spinning, the C-arm support 12 can operate in a two-dimensional or two-degree motion (e.g., around two perpendicular shafts). As a result, in operation, the C-arm imaging system 10 is equipped with the spherical maneuverability of the C-arm support 12. For example, due to the sliding rail motion and side spinning, an X-ray source 32 coupled to the C-arm support 12 can be moved to any substantial latitude/longitude point of the lower hemisphere of the imaginary ball when the C-arm support 12 moves around the same.

The C-arm imaging system 10 includes the X-ray source 32 and an image receiver 34, which are respectively mounted at opposite positions on the C-arm support 12. The X-ray source 32 and the image receiver 34 may collectively be referred to as an X-ray source/image receiver. The image receiver 34 may be an image enhancer or another light enhancing member. The rail and side spinning operation of the C-arm support 12 enables the X-ray source 32 and the image receiver 34 to be oppositely located at the width and the length positions of the patient in an inner space of the C-arm support 12 for selective positioning. Expressly, the C-arm X-ray imaging system 10 may include a servo system coupled to a controller 38 (e.g., a digital/electrical/mechanical system performing mechanical motion under software control, which may use feedback) The sliding rail motion of the C-arm support 12 enables the X-ray source 32 and the image receiver 34 to move along the respective arc-shaped paths. In one exemplary embodiment, the image receiver 34 is fixed to the inner circumference 14 of the C-arm support 12, and the X-ray source 32 can also be fixed to the inner circumference 14.

It should be noted that additional or different constituent parts may be provided as a part of the C-arm imaging system 10. For example, the C-arm imaging system 10 can include a workbench used for supporting the patient in a space 36. In operation, in order to generate an image of the patient, the C-arm support 12 is rotated so that the X-ray source 32 and the image receiver 34 are moved around the patient. Expressly, the C-arm support 12 is rotatably coupled to the support member 20 such that the X-ray source 32 and the image receiver 34 rotate around the patient or other objects to be imaged.

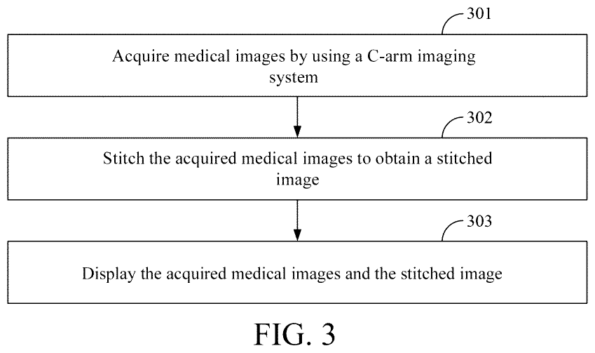

FIG. 2 is a schematic diagram of a control system for controlling the C-arm imaging system as shown in FIG. 1, schematically showing a functional block diagram of the control system. As shown in FIG. 2, the movement of the C-arm support 12 and the operation of the X-ray source 32 and the image receiver 34 are controlled by a control mechanism 52 of the C-arm imaging system 10, which may be provided as a part of the controller 38 (as shown in FIG. 1). The control mechanism 52 generally includes an X-ray controller 54 and a motor controller 56. The X-ray controller 54 provides power and a timing signal to the X-ray source 32. The motor controller 56 controls the positions of the C-arm support 12, the X-ray source 32, and the image receiver 34.

In one exemplary embodiment, a data acquisition system (DAS) 58 provided as a part of the control mechanism 52 performs sampling on data from the image receiver 34 (such as an X-ray detector) for subsequent processing. An image processor/reconstituting unit 60 receives the sampled X-ray data from the DAS 58 and performs image processing/reconstitution. A generated image is provided as an input to a computer 62, and the computer 62 may store the image in a mass storage device 63 (such as a disk memory). It should be pointed out that the term “reconstituting unit” as used herein includes reconstituting units known in the field of medical imaging and other suitable processes for processing data collected during scanning.

The computer 62 also receives commands and scanning parameters from an operator by means of a console 64 that includes user inputs (such as a keyboard). One or more displays 66 allow the operator to observe the obtained image and other data from the computer 62. The commands and parameters provided by the operator are used by the computer 62 to provide control signals and information to the DAS 58, the X-ray controller 54, and the motor controller 56. The computer 62 also operates a workbench motor controller 68, and the workbench motor controller 68 can control, for example, the position of an electric workbench (not shown) relative to the C-arm imaging system 10 (as shown in FIG. 1).

A medical imaging system is schematically illustrated above by taking the C-arm imaging system as an example, but the present application is not limited thereto. The medical imaging system may be any other suitable medical imaging devices. A storage device (mass storage device 63) may be located within the medical imaging system, in a server outside the medical imaging system, in an independent medical image storage system (such as a picture archiving and communication system (PACS)), and/or in a remote cloud storage system.

As an example, after a medical institution completes an imaging scan using the medical imaging apparatus, scan data is stored in the storage apparatus (mass storage device 63). The medical imaging workstation may directly read the scan data and perform image processing by means of a processor thereof. As another example, the medical image cloud platform analysis system may read a medical image in the storage apparatus by means of remote communication to provide “software as a service (SAAS).” SAAS can exist between hospitals, between a hospital and an imaging center, or between a hospital and a third-party online diagnosis and treatment service provider.

The medical imaging device is schematically illustrated above, and the embodiments of the present application are described in detail below with reference to the accompanying drawings.

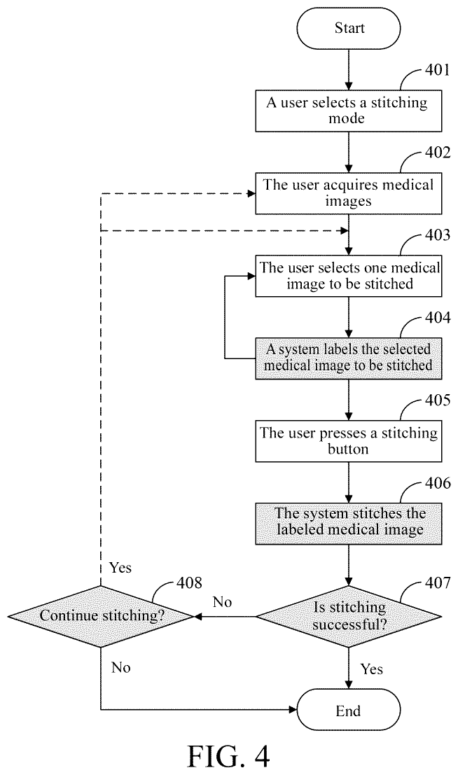

Provided in an embodiment of the present application is a medical image processing method applied to a C-arm imaging system. FIG. 3 is a schematic diagram of a medical image processing method according to the embodiment of the present application. As shown in FIG. 3, the method includes step 301, acquiring medical images by using the C-arm imaging system; step 302, stitching the acquired medical images to obtain a stitched image; and step 303, displaying the acquired medical images and the stitched image.

It should be noted that FIG. 3 merely schematically illustrates the embodiment of the present application, but the present application is not limited thereto. For example, some other operations may also be added or some of these operations may be omitted. Those skilled in the art could make appropriate variations according to the above disclosure, rather than being limited by the disclosure of FIG. 3.

According to the embodiment of the present application, an image stitching function suitable for a C-arm imaging system can be provided for the C-arm imaging system, thereby providing reference for a doctor to evaluate surgical effects during or after surgery.

In step 301 described above, the acquired medical images may be stored automatically in a memory of the C-arm imaging system, and the mode of acquiring and the form of storage are not limited in the present application, and the related art can be referred to. For example, the patient or other objects to be imaged may be photographed or exposed using the X-ray source 32 and the image receiver 34 of the C-arm imaging system 10 as shown in FIG. 1 and the acquired data is reconstructed by an image processor 60 of the control system as shown in FIG. 2 to acquire medical images of the patient or other objects to be imaged, and the medical image may also be stored by the mass storage device 63 as shown in FIG. 2. According to the surgical needs, a plurality of medical images can be obtained by rotating the C-arm support 12.

In the embodiments of the present application, reconstruction and storage of the medical images are performed synchronously for simplicity of illustration and convenience of understanding. In the illustration below, “acquired medical images” refer to medical images acquired and reconstructed and stored.

In some of the embodiments of the present application, in step 302, image stitching is performed based on the medical images that have been acquired. Here, the number of the medical images that have been acquired is at least two, and this stitching mode is referred to as a first stitching mode. Thus, by labeling the medical images selected from the acquired medical images, and stitching the labeled medical images, a stitched image is obtained, and the stitched image as a panoramic image can provide reference for the doctor to evaluate the surgery. In order to improve the efficiency of image stitching, a plurality of images to be selected (that is, the plurality of acquired medical images) can also be pre-screened, e.g., preliminary screening can be performed in an AI (artificial intelligence) manner to screen off images that are significantly ineligible for stitching.

Further, to improve the efficiency of user selection of the medical images, before selection by the user, a prompt for the photographing location and/or photographing coordinates may also be displayed for the plurality of images to be selected, e.g., the location or coordinates of the site where the current image is photographed may be determined based on image identification or AI, or prompts may be provided with respect to the plurality of images in the order of the photographed sites (e.g., the order from the base of the thigh to the heel). More intuitively, the plurality of images may be displayed in sequence based on the order of the photographed sites, and the user may be provided with an intuitive indication through the prompts to facilitate selection by the user.

For example, during the surgery, by rotating the C-arm support 12 to acquire the plurality of medical images, the user (e.g., a doctor) may select medical images desired for image stitching from the plurality of acquired medical images when the first stitching mode is activated.

The acquired medical images may all be displayed on a display interface of the C-arm imaging system, for example, may be displayed by the display 66 of the control system as shown in FIG. 2. The user selects the medical images desired to be stitched (the images to be stitched) by operating the medical images displayed on the display interface, and at this moment, all or part of the acquired medical images are used for stitching.

For example, the user may select the stitching mode and the image to be stitched by the operator console 64 of the control system as shown in FIG. 2; or, if the display 66 has a touch function, the user may also perform operation directly on the display 66 to select the stitching mode and the image to be stitched.

In the above embodiment, the medical image selected by the user may be labeled, whereby the user may intuitively know which images participate in image stitching (i.e., generation of the panoramic image).

The labeling manner is not limited in the present application, e.g., the selected medical image may be labeled by displaying a label on the acquired medical image, or, the selected medical image may also be labeled by displaying the selected medical image in a particular region, or the selected medical image may also be labeled by blurring the selected medical image, or the like.

Further, in the label displaying manner, the position of the label is not limited in the present application, e.g., the label may be superimposed and displayed at the lower right corner or the upper left corner or other positions of an original medical image. In addition, in the label displaying manner, the form of the label is not limited in the present application, may be a tick (V), or a cross (x), or labels in other arbitrary forms.

In the above embodiments, the selected medical images may be labeled according to the selecting order of the medical images and the labeled medical images are stitched according to the labeling order (i.e., the selecting order). For example, one medical image is labeled once the user selects one medical image, after all the selected medical images are labeled, and/or, when the stitching function is activated, the labeled medical images are stitched according to the labeling order (i.e., the selecting order).

In the embodiment of the present application, since the medical images are acquired by operating the C-arm imaging system (e.g., the C-arm support 12 described above), not all the acquired medical images are necessary for the surgery by the doctor, or some acquired medical images may be fuzzy or improper in position, or the like. In accordance with the method of the embodiment of the present application, the doctor may exclude some medical images by manual selection, thereby reducing the calculating amount of image stitching and improving the efficiency of image stitching, and a panoramic image obtained accordingly is more in line with the expectations of the doctor, and can provide reference for the doctor to evaluate surgical effects.

In the above embodiments, if the majority of the acquired medical images cannot meet the needs of the doctor, e.g., the doctor cannot select suitable medical images for forming a panoramic image from the acquired medical images, then acquisition needs to be performed again e.g., re-operating the C-arm support 12 for acquiring medical images. In this way, the burden on patients is undoubtedly increased, which is not conducive to smooth surgery. Thus, in some embodiments, it is possible to acquire only a small number of medical images for stitching. If the small number of medical images are sufficient to complete stitching, the C-arm support 12 continues to be operated to acquire medical images at a next location, and the medical images continuing to be acquired and selected are stitched onto a previous result; if the small number of medical images are not sufficient to complete stitching, the C-arm support 12 is operated at the current position to continue to perform the operation of acquiring, selecting (labeling), and stitching medical images. Thus, the risk of repeated acquisition can be reduced.

In the above embodiment, stitching the labeled medical images comprises determining whether the labeled images are sufficient to complete stitching according to a preset condition, and then generating the aforementioned stitched image if it is determined that stitching can be completed.

Here, the preset condition may be set as desired, for example, the condition may be whether the overlap area of two adjacent images participating in stitching (labeled images) is less than a threshold. If the overlap area is less than the threshold, it indicates that the distance of the photographing locations corresponding to the two images is relatively far, and it may not be possible to reflect the real condition of the patient if the two images are stitched, and then it is considered that the two images cannot be used to complete stitching, i.e., stitching failure. For another example, the condition may be whether a matching feature point of two adjacent images participating in stitching (labeled images) is less than a threshold. If the matching feature point is less than the threshold, that is, the matching feature point of the two images is smaller, that is, the two images are not matched, then it is considered that the two images cannot be used to complete stitching, i.e., stitching failure.

In the above example, the calculation and determination of the feature point is not limited, and the feature point of two adjacent images participating in stitching may be calculated according to an existing method, and then it is determined whether the matching feature point of the two is less than the threshold.

In addition, the preset condition is illustrated above only by using the overlap area and matching feature point as examples, but the present application is not limited thereto. Other conditions may also be set in accordance with different surgical categories or different demands of the doctor, so as to determine whether medical images participating in stitching (labeled medical images) can be used to complete stitching, and the description thereof is omitted here.

In the above embodiment, if it is determined that stitching cannot be completed, medical images may be acquired again or re-selected medical images may be re-labeled, thereby continuing the subsequent stitching procedure; or the stitching procedure may be terminated according to the user's operation.

For example, if it is determined that stitching cannot be completed, then whether to continue stitching is determined, and the determination can be completed according to the user's operation. When the user selects to continue stitching, medical images may be acquired again by means of continuing to use the above-mentioned C-arm imaging system and labeling medical images re-selected by the user, and then the subsequent stitching procedure continues. Alternatively, when the user selects to continue stitching, the medical images re-selected by the user may continue to be labeled to continue the subsequent stitching procedure. In the above example, if the determination is no, that is, the user selects not to continue stitching, then the stitching procedure ends, the stitched image is output or it is considered that stitching fails, and the stitching procedure is restarted according to the user's operation.

In the above embodiment, the stitched image can also be displayed on a display interface of the C-arm imaging system, for example, displayed by the display 66 of the control system as shown in FIG. 2.

In some embodiments, the stitched image may be displayed in the first region of the display interface described above, and all the acquired medical images and the labeled medical images may be displayed in the second region of the display interface described above.

In the above embodiments, due to the limitations of the display area, the above acquired medical images and the labeled medical images may be displayed as thumbnail images, or may also be displayed in a multi-page manner, and the specific display manner is not limited in the present application.

In the above embodiment, the stitched image as a panoramic image provides reference for the doctor, and thus, the area of the first region may be greater than the area of the second region, but the present application is not limited thereto. Further, the stitched image may also be displayed as a thumbnail image, and may be displayed as an original image in accordance with the user's operation. The related art may be referred to for specific display control methods, and the description thereof is omitted here.

In the embodiments described above, after the stitched image is obtained, the stitched image may also be post-processed, e.g., the brightness and contrast of the stitched image may be adjusted, and the stitched image may also be saved, printed, or exported. The stitched image as a panoramic image can provide reference for the doctor to evaluate surgical effects.

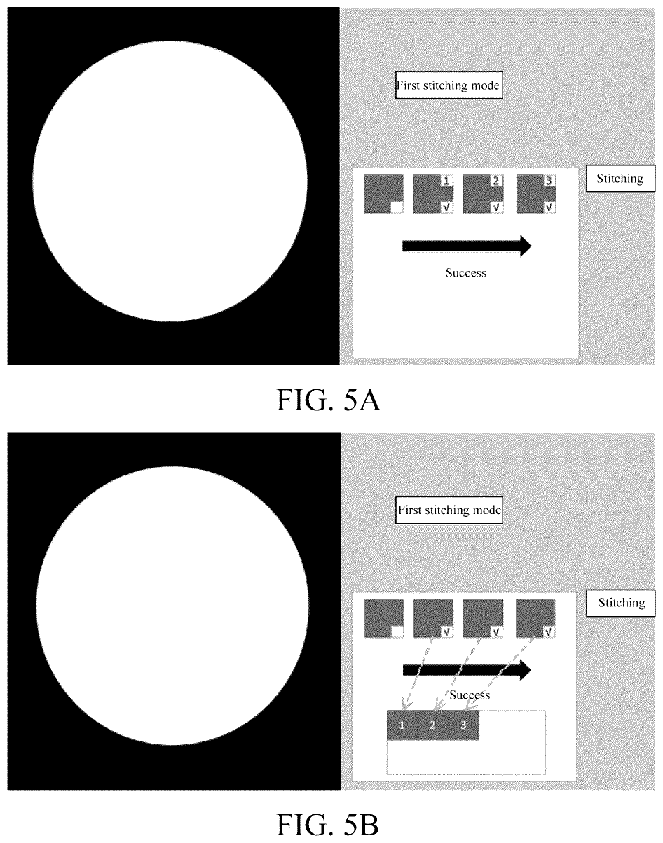

FIG. 4 is an overall schematic view of a stitching procedure in a first stitching mode, including operation of the user and operation of the system, wherein the operation of the system is implemented by the method of the embodiment of the present application. As shown in FIG. 4, the process includes step 401, the user selecting a stitching mode (first stitching mode); step 402, the user acquiring medical images, the medical images being automatically stored into a memory; step 403, the user selecting a medical image to be stitched; step 404, the system labeling the selected medical image to be stitched; step 405, the user pressing a stitching button; step 406, the system starting to stitch the labeled medical image; step 407, determining whether stitching succeeds, ending the stitching procedure if so, and performing step 408 if not; step 408, determining whether to continue stitching, if so, returning to step 402 to acquire images again or returning to step 403 to re-select images, and if not, ending the stitching procedure.

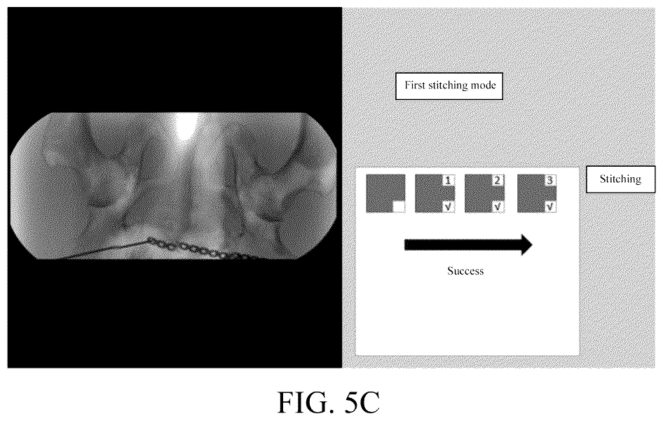

FIGS. 5A-5C are schematic diagrams for stitching medical images selected by the user in the first stitching mode, and show the case of acquiring four medical images.

As shown in FIG. 5A, a left-side region (first region) of the display interface is used for displaying the stitched image, and a right-side region (second region) of the display interface is used for displaying the acquired images and the labeled images. In this example, the acquired images are displayed in the right-side region as thumbnail images, and the method of the present application labels images selected by the user, and in the example of FIG. 5A, the method of the present application performs labeling in a manner of adding a tick (I) to the lower right corner of the image. However, the present application does not limit the manner and form of labeling.

Further, in the example of FIG. 5A, the method of the present application also labels the order in which the user selects the images by means of the serial numbers “1, 2, 3” at the upper right corner of the labeled images. Likewise, the present application does not limit the manner and form of labeling the order in which the user selects the image.

As shown in FIG. 5B, unlike the example of FIG. 5A, the images selected by the user are sequentially arranged in a region in the second region. In this way, it is equivalent to labeling the images selected by the user, and the order of selecting is labeled.

As shown in FIG. 5C, in the method of the present application, after the user presses the stitching button, stitching of the labeled images is started, and if it is determined that stitching can be completed, or if it is determined that stitching succeeds, the stitched image is displayed in the first region (the left-side region of the display interface as shown in FIG. 5C).

In other embodiments of the embodiments of the present application, in step 302, image stitching is performed based on the medical images acquired in real time; here, the number of the medical images acquired in real time is at least one, and such a stitching mode is referred to as a second stitching mode. Thus, by stitching the at least one medical image acquired in real time in the order of acquiring, the stitched image is obtained, and the stitched image as a panoramic image may provide reference for the doctor to evaluate the surgery.

For example, during the surgery, when the second stitching mode is activated, by rotating and/or moving the C-arm support 12 to acquire and save the medical images in real time for the user (such as a doctor), after acquiring one medical image, the user presses the stitching button, thereby stitching each of the medical images acquired subsequently in real time with the saved medical image, so that continuous stitching is achieved.

According to the method of the embodiment of the present application, the user only needs to perform one operation and can complete continuous stitching of the medical image, which further improves the efficiency of image stitching.

In the above embodiment, stitching the medical images acquired in real time may include determining whether the medical images acquired in real time can be used to complete stitching according to the preset condition, and generating the aforementioned stitched image if it is determined that stitching can be completed. Here, the preset condition has been illustrated above, the contents of which are incorporated herein, and will not be described again.

In the above embodiment, if it is determined that stitching cannot be completed, medical images may be acquired again at the current location, and then the subsequent stitching procedure continues; or the stitching procedure may also be terminated according to the user's operation.

For example, if it is determined that stitching cannot be completed, then whether to continue stitching is determined, and the determination can be completed according to the user's operation. When the user selects to continue stitching, medical images can be acquired again by means of continuing to use the above-mentioned C-arm imaging system, and then the subsequent stitching procedure continues. In the above example, if the determination is no, that is, the user selects not to continue stitching, then the stitching procedure ends, the stitched image is output or it is considered that stitching fails, and the stitching procedure is restarted according to the user's operation.

In the above embodiment, the medical images acquired in real time may be displayed on the display interface of the C-arm imaging system, for example, displayed by the display 66 of the control system as shown in FIG. 2. Further, the stitched image can also be displayed on the display interface of the C-arm imaging system, for example, displayed by the display 66 of the control system as shown in FIG. 2. The display manner of the acquired medical images and the stitched images has been explained above, the contents of which are incorporated herein, and will not be described again.

In the above embodiments, the medical images participating in stitching may also be labeled, e.g., the medical images participating in image stitching are labeled after completing stitching, whereby the user may intuitively know which medical images participate in image stitching (i.e., generation of the panoramic image).

The labeling manner is not limited in the present application, e.g., the medical image participating in stitching may be labeled by displaying a label on the medical image participating in stitching, or the medical image participating in stitching may also be labeled by displaying the medical image participating in stitching in a particular region, or the medical image participating in stitching may also be labeled by blurring the medical image participating in stitching, or the like.

Further, in the label displaying manner, the position of the label is not limited in the present application, e.g., the label may be superimposed and displayed at the lower right corner or the upper left corner or other positions of an original medical image. In addition, in the label displaying manner, the form of the label is not limited in the present application, may be a tick (V), or a cross (x), or labels in other arbitrary forms.

In the above embodiments, the stitching order of the medical image participating in stitching may also be labeled. For example, after a certain medical image acquired in real time is stitched, the stitching order thereof is labeled by serial numbers or other means.

In the embodiments described above, after the stitched image is obtained, the stitched image may also be post-processed, e.g., the brightness and contrast of the stitched image may be adjusted, and the stitched image may also be saved, printed, or exported. The stitched image as a panoramic image can provide reference for the doctor to evaluate surgical effects.

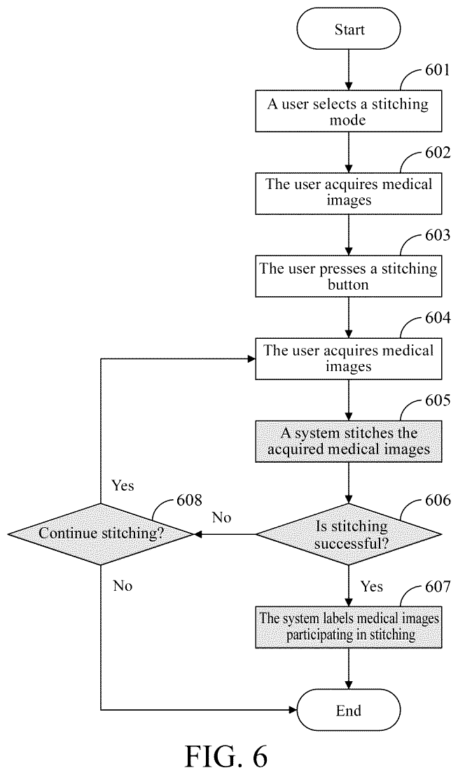

FIG. 6 is an overall schematic view of a stitching procedure in a second stitching mode, including operation of the user and operation of the system, wherein the operation of the system is implemented by the method of the embodiment of the present application. As shown in FIG. 6, the process includes step 601, the user selecting a stitching mode (second stitching mode); step 602, the user acquiring medical images, for example, acquiring m medical images, the m medical images being stored automatically in a memory, where m≥1; step 603, the user pressing a stitching button; step 604, the user acquiring the medical images, for example, acquiring n medical images, where n≥1; step 605, the system stitching the acquired medical images, e.g., stitching the m medical images and the n medical images; step 606, determining whether stitching succeeds, performing step 607 if so, and performing step 608 if not; step 607, the system sequentially labeling medical images participating in stitching; and step 608, determining whether to continue stitching, if so, returning to step 604 to continue to acquire images, and if not, ending the stitching procedure.

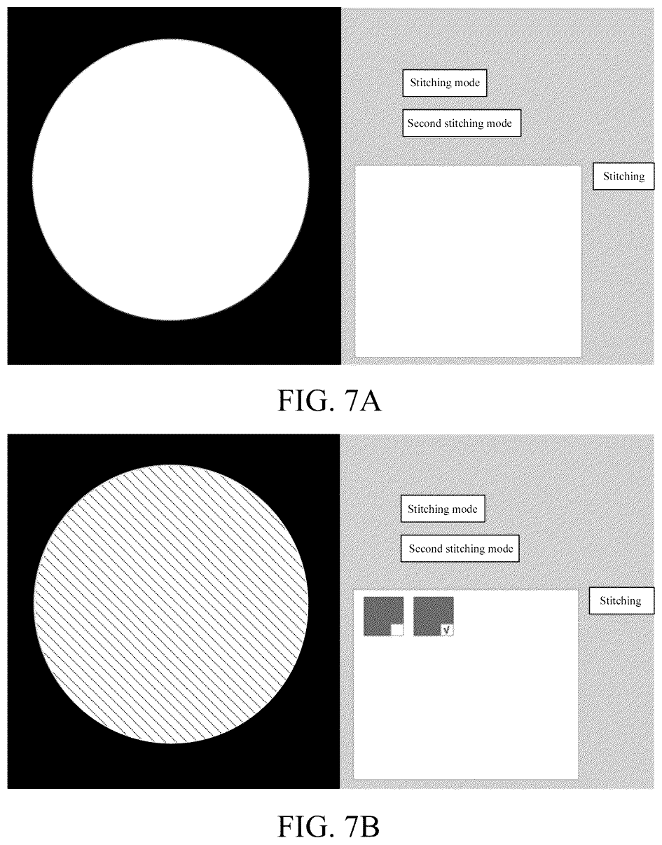

FIGS. 7A-7D are schematic diagrams for stitching medical images acquired in real time by the user in the second stitching mode.

As shown in FIG. 7A, a left-side region (first region) of the display interface is used for displaying the stitched image, and a right-side region (second region) of the display interface is used for displaying the acquired images and the labeled images. When the user selects the second stitching mode and presses the stitching button, continuous stitching then starts.

As shown in FIG. 7B, the acquired images are displayed in the right-side region as thumbnail images, and in the method of the present application, the image participating in stitching is labeled. In the example of FIG. 7B, labeling is performed in the method of the present application in a manner of adding a tick (I) to the lower right corner of the image participating in stitching. However, the present application does not limit the manner and form of labeling. In the example of FIG. 7B, there is one image participating in stitching.

As shown in FIG. 7C, the acquired images are displayed in the right-side region as thumbnail images, and in the method of the present application, the image participating in stitching is labeled. As shown in FIG. 7C, in the method of the present application, labeling is performed in a manner of adding a tick (I) to the lower right corner of the image participating in stitching. In the method of the present application, the stitching order of the image participating in stitching is labeled, and as shown in FIG. 7C, in the method of the present application, the stitching order of the image participating in stitching is labeled in a manner of labeling serial numbers at the upper right corner of the image participating in stitching. In the example of FIG. 7C, there are two images participating in stitching, and the currently acquired image P1 also completes stitching, and labeling about whether to participate in stitching and labeling of the stitching order are performed. At this moment, it can be determined to continue stitching or to end stitching according to the user's operation. For example, if the user operates a “stop” button, the stitching procedure ends; if the user does not operate the “stop” button, the method of the present application continues to stitch a next image (image at the next position) acquired in real time.

As shown in FIG. 7D, the acquired images are displayed in the right-side region as thumbnail images, unlike the example of FIG. 7C, the currently acquired image P2 does not complete stitching, i.e., stitching failure, and thus it is not labeled. At this moment, it can be determined to continue stitching or to end stitching according to the user's operation. For example, if the user operates the “stop” button, the stitching procedure ends; if the user does not operate the “stop” button, the method of the present application continues to stitch images acquired in real time (which may be images at the current position and may also be images at the next position).

In the example of FIGS. 7B-7D, the image participating in stitching is labeled with a tick (I) and the order of the image participating in stitching is labeled with the serial number, but the present application is not limited thereto. The image participating in stitching and the order of the image participating in stitching may also be labeled in other ways, and the description is omitted herein.

In the embodiments of the present application, switching control may also be performed on the two stitching modes described above, e.g., when the user selects the first stitching mode, the control system performs the workflow in the first stitching mode described above, when the user selects the second stitching mode, the control system performs the workflow in the second stitching mode described above, and the present application does not limit the specific control manner.

Embodiments of the present application further provide a method for using a C-arm imaging system, the method including step S1, moving the C-arm imaging system such that the C-arm imaging system acquires medical images at a plurality of positions; step S2, operating a stitching mode selecting button to select a stitching mode; and step S3, operating a stitching button such that the C-arm imaging system stitches the acquired medical images to obtain a stitched image and displays the acquired medical images and the stitched image.

It should be noted that the above merely schematically illustrates the embodiment of the present application, but the present application is not limited thereto. For example, the order of execution between various steps may be suitably adjusted. In addition, some other steps may also be added or some of these steps may be omitted. Those skilled in the art could make appropriate variations according to the above disclosure, rather than being limited by the above descriptions.

For example, step S1 may be performed first such that the C-arm imaging system acquires the medical images at a plurality of positions, then step S2 is performed to select the stitching mode, if the selected stitching mode is a first stitching mode, step S3 is performed to enable the C-arm imaging system to stitch the acquired medical images by operating a stitching button; if the selected stitching mode is a second stitching mode, step S1 continues to be performed after step S3 is performed to acquire medical images in real time, and then step S3 continues to be performed for image stitching.

For another example, step S2 can be performed first to select a stitching mode, if the selected stitching mode is the first stitching mode, step S1 is performed to enable the C-arm imaging system to acquire medical images at a plurality of positions, and finally step S3 is performed to enable the C-arm imaging system to stitch the acquired medical images by operating a stitching button; if the selected stitching mode is the second stitching mode, step S3 is performed, and after the stitching button is operated, step S1 is performed to acquire medical images in real time, and stitching operation is performed on the medical images acquired in real time.

In the above embodiments, the process and display manner of image stitching in different stitching modes has been explained in detail above, and will not be described herein again.

The above embodiments merely provide illustrative description of the embodiments of the present application. However, the present application is not limited thereto, and appropriate variations may be made on the basis of the above embodiments. For example, each of the above embodiments may be used independently, or one or more of the above embodiments may be combined.

According to the embodiment of the present application, an image stitching function suitable for the C-arm imaging system can be provided for the C-arm imaging system, thereby providing reference for a doctor to evaluate surgical effects during or after surgery.

Provided in an embodiment of the present application is a C-arm imaging system, the C-arm imaging system may include the composition as shown in FIG. 1 and FIG. 2. The related art may be referred to for the specifics thereof, which will not be described again herein.

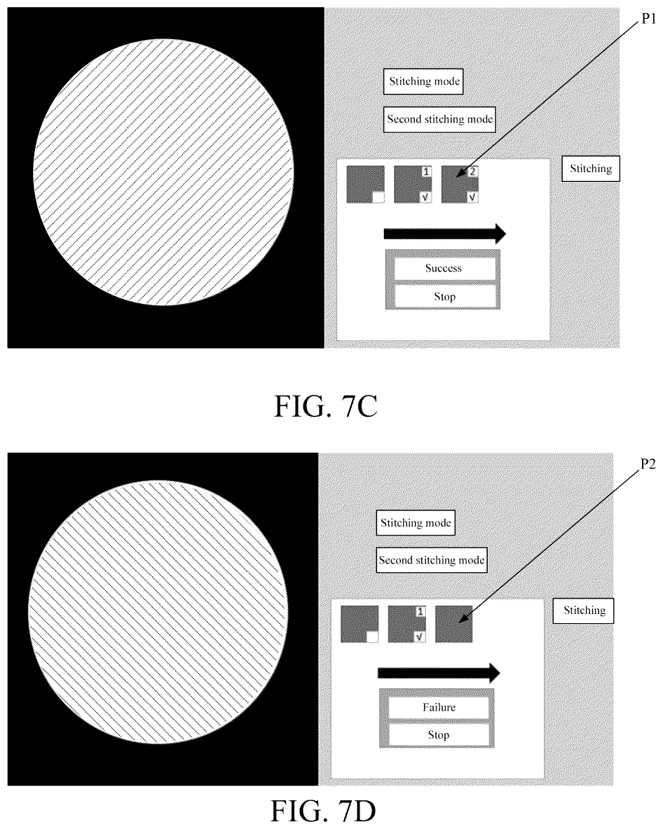

FIG. 8 is another schematic diagram of the C-arm imaging system according to an embodiment of the present application. As shown in FIG. 8, a C-arm imaging system 800 according to an embodiment of the present application further includes an acquisition unit 801 for acquiring medical images; a first stitching unit 802 for labeling, in a first stitching mode, medical images selected from the acquired medical images, and stitching the labeled medical images to obtain a stitched image; and a display unit 803 for respectively displaying the acquired medical images, the labeled medical images, and the stitched image.

In some embodiments, stitching the labeled medical images by the first stitching unit 802 comprises determining whether stitching can be completed according to a preset condition; if it is determined that stitching can be completed, generating the stitched image.

In the above embodiment, stitching the labeled medical images by the first stitching unit 802 further comprises if it is determined that stitching cannot be completed, determining whether to acquire medical images again; if it is determined that medical images are required to be acquired again, labeling medical images re-selected from the acquired medical images, and stitching the re-labeled medical images.

In another embodiment, the first stitching unit 802 labels the selected medical images according to the selecting order of the medical images and stitches the labeled medical images according to the labeling order.

In some embodiments, as shown in FIG. 8, the system 800 further includes a second stitching unit 804, configured to stitch, in a second stitching mode, one or more medical images acquired in real time by the acquisition unit 801 according to the acquiring order, so as to obtain a stitched image.

In the above embodiment, the second stitching unit 804 further labels the stitched medical images.

In the above embodiment, stitching, by the second stitching unit 804, the medical images acquired in real time comprises determining whether stitching can be completed according to a preset condition; if it is determined that stitching can be completed, generating the stitched image.

In the above embodiment, stitching, by the second stitching unit 804, the medical images acquired in real time further comprises if stitching ends, outputting the stitched image; and if stitching does not end, stitching, by the acquisition unit 801, medical images acquired again in real time.

In the above embodiments, as shown in FIG. 8, the system 800 further includes a control unit 805 for performing switching control to switch between the first stitching mode and the second stitching mode.

In some embodiments, the display unit 803 displays the stitched image in a first region, and displays the acquired medical images and the labeled medical images in a second region as thumbnail images.

In the above embodiment, the acquisition unit 801 may be implemented by means of the X-ray source 32 and the image receiver 34 as shown in FIG. 1 and the image processor 60 as shown in FIG. 2, and the acquired images may be stored in the mass storage device 63 as shown in FIG. 2. The first stitching unit 802, the second stitching unit 804, and the control unit 805 may be implemented by means of the computer 62 as shown in FIG. 2. The display unit 803 may be implemented by means of the computer 62 and the display 66 as shown in FIG. 2. The function of the relevant composition has been explained in detail above, the contents of which are incorporated herein, and will not be described again.

For the sake of simplicity, FIG. 8 only exemplarily illustrates a connection relationship or signal direction between various components or modules, but it should be clear to those skilled in the art that various related technologies such as bus connection can be used. The various components or modules can be implemented by means of a hardware facility such as a processor, a memory, etc. The embodiments of the present application are not limited thereto.

The above embodiments merely provide illustrative description of the embodiments of the present application. However, the present application is not limited thereto, and appropriate variations may be made on the basis of the above embodiments. For example, each of the above embodiments may be used independently, or one or more of the above embodiments may be combined.

According to the embodiment of the present application, an image stitching function suitable for the C-arm imaging system can be provided for the C-arm imaging system, thereby providing reference for a doctor to evaluate surgical effects during or after surgery.

Provided in an embodiment of the present application is an electronic device, including a medical image processing device 800 as described in the embodiment of the second aspect, the contents of which are incorporated herein. The electronic device may, for example, be a computer, a server, a workstation, a laptop, a smart phone, etc., but the embodiments of the present application are not limited thereto.

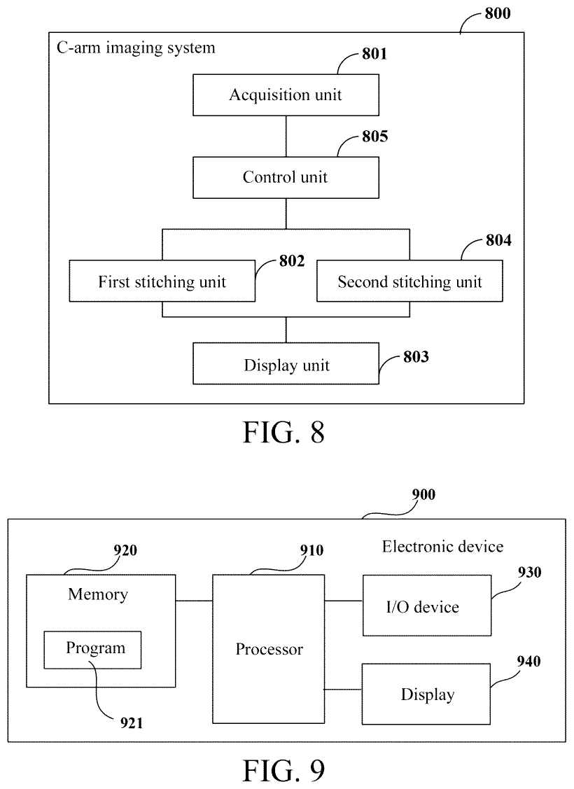

FIG. 9 is a schematic diagram of an electronic device according to an embodiment of the present application. As shown in FIG. 9, an electronic device 900 may include one or more processors (e.g., a central processing unit (CPU)) 910 and one or more memories 920 coupled to the processors 910. The memory 920 can store various data, further store a program 921 for information processing, and executes the program 921 under control of the processor 910.

In some embodiments, functions of the medical image processing device 800 are integrated into and implemented by the processor 910. The processor 910 is configured to implement the medical image processing method according to the embodiments of the first aspect.

In some embodiments, the medical image processing device 800 and the processor 910 are configured separately, for example, the medical image processing device 800 can be configured as a chip connected to the processor 910 and the functions of the medical image processing device 800 can be achieved by means of the control of the processor 910.

For example, the processor 910 is configured to perform the following control acquiring medical images by using the C-arm imaging system; in a first stitching mode, labeling medical images selected from the acquired medical images, and stitching the labeled medical images to obtain a stitched image; and respectively displaying the acquired medical images, the labeled medical images, and the stitched image.

In addition, as shown in FIG. 9, the electronic device 900 may further include an input/output (I/O) device 930, a display 940, etc. Functions of the above components are similar to those in the prior art, and are not repeated herein. It should be noted that the electronic device 900 does not necessarily include all of the components shown in FIG. 9. In addition, the electronic device 900 may further include components not shown in FIG. 9, for which reference may be made to the related technologies.

Embodiments of the present application further provide a computer-readable program, wherein when the program is executed in an electronic device, the program enables a computer to execute, in the electronic device, the medical image processing method as described in the embodiment of the first aspect.

Embodiments of the present application further provide a storage medium for storing a computer-readable program, wherein the computer-readable program enables a computer to execute, in the electronic device, the medical image processing method as described in the embodiment of the first aspect.

The above device and method of the present application can be implemented by hardware, or can be implemented by hardware in combination with software. The present application relates to such a computer-readable program, when executed by a logical component, causes the logical component to implement the foregoing device or constituent part, or causes the logical component to implement various methods or steps as described above. The present application further relates to a storage medium for storing the above program, such as a hard disk, a magnetic disk, an optical disk, a DVD, a flash memory, etc.

The method/device described with reference to the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. These software modules may respectively correspond to the steps shown in the figures. These hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any storage medium in other forms known in the art. A storage medium may be coupled to a processor, so that the processor can read information from the storage medium and can write information into the storage medium. Alternatively, the storage medium may be a component of the processor. The processor and the storage medium may be located in an ASIC. The software module may be stored in a memory of a mobile terminal, and may also be stored in a memory card that can be inserted into a mobile terminal. For example, if an apparatus (such as a mobile terminal) uses a large-capacity MEGA-SIM card or a large-capacity flash memory device, the software modules can be stored in the MEGA-SIM card or the large-capacity flash memory device.

One or more of the functional blocks and/or one or more combinations of the functional blocks shown in the accompanying drawings may be implemented as a general-purpose processor, a digital signal processor (DSP), an application specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing apparatuses, such as a combination of a DSP and a microprocessor, multiple microprocessors, one or more microprocessors in communication combination with a DSP, or any other such configuration.

The present application is described above with reference to specific implementations. However, it should be clear to those skilled in the art that such description is merely illustrative and is not intended to limit the scope of protection of the present application. Various variations and modifications could be made by those skilled in the art according to the principle of the present application, and these variations and modifications also fall within the scope of the present application.

What is claimed is:

1. A C-arm imaging system, characterized by comprising:
an acquisition unit for acquiring medical images;
a first stitching unit for labeling, in a first stitching mode, medical images selected from the acquired medical images, and stitching the labeled medical images to obtain a stitched image; and
a display unit for respectively displaying the acquired medical images, the labeled medical images, and the stitched image.

2. The system according to claim 1, wherein stitching the labeled medical images by the first stitching unit comprises:
determining whether stitching can be completed according to a preset condition; and
if it is determined that stitching can be completed, generating the stitched image.

3. The system according to claim 2, wherein stitching the labeled medical images by the first stitching unit further comprises:
if it is determined that stitching cannot be completed, determining whether to acquire medical images again; and
if it is determined that medical images are acquired again, labeling medical images re-selected from the acquired medical images, and stitching the re-labeled medical images.

4. The system according to claim 3, wherein the display unit displays the stitched image in a first region, and displays the acquired medical images and the labeled medical images in a second region as thumbnail images.

5. The system according to claim 1, wherein the first stitching unit labels the selected medical images according to the selecting order of the medical images and stitches the labeled medical images according to the labeling order.

6. The system according to claim 5, wherein the display unit displays the stitched image in a first region, and displays the acquired medical images and the labeled medical images in a second region as thumbnail images.

7. The system according to claim 1, further comprising a second stitching unit for stitching, in a second stitching mode, one or more medical images acquired in real time by the acquisition unit according to the acquiring order, so as to obtain a stitched image.

8. The system according to claim 7, wherein the display unit displays the stitched image in a first region, and displays the acquired medical images and the labeled medical images in a second region as thumbnail images.

9. The system according to claim 5, wherein the second stitching unit further labels the stitched medical images.

10. The system according to claim 5, wherein stitching, by the second stitching unit, the medical images acquired in real time comprises:
determining whether stitching can be completed according to a preset condition; and
if it is determined that stitching can be completed, generating the stitched image.

11. The system according to claim 7, wherein stitching, by the second stitching unit, the medical images acquired in real time further comprises:
if stitching ends, outputting the stitched image; and
if stitching does not end, stitching medical images acquired again in real time.

12. The system according to claim 11, wherein the display unit displays the stitched image in a first region, and displays the acquired medical images and the labeled medical images in a second region as thumbnail images.

13. The system according to claim 5, further comprising a control unit for performing switching control to switch between the first stitching mode and the second stitching mode.

14. The system according to claim 13, wherein the display unit displays the stitched image in a first region, and displays the acquired medical images and the labeled medical images in a second region as thumbnail images.

15. A medical image processing method, applied to a C-arm imaging system, and characterized by comprising:
acquiring medical images by using the C-arm imaging system;
in a first stitching mode, labeling medical images selected from the acquired medical images, and stitching the labeled medical images to obtain a stitched image; and
respectively displaying the acquired medical images, the labeled medical images, and the stitched image.

16. The method according to claim 15, wherein stitching the labeled medical imagines comprises:
determining whether stitching can be completed according to a preset condition; and
if it determined to be completed, generating the stitched image.

17. A method for using a C-arm imaging system, characterized in that the method comprises:
moving the C-arm imaging system such that the C-arm imaging system acquires medical images at a plurality of positions;
operating a stitching mode selecting button to select a stitching mode; and
operating a stitching button such that the C-arm imaging system stitches the acquired medical images to obtain a stitched image and displays the acquired medical images and the stitched image.

18. The method of claim 17, further comprising stitching in a second stitching mode, one or more medical images acquired in real time by the C-arm imaging system according to the acquiring order so as to obtain a stitched image.

19. The method of claim 18, further comprising labeling the stitched medical image.

20. The method according to claim 18, wherein stitching the medical images acquired in real time comprises:
determining whether stitching can be completed according to a preset condition; and
if it is determined that stitching can be completed, generating the stitched image.
